(19) United States
(12) Patent Application Publication
(10) Pub. No.: US 2007/0202205 A1
(43) Pub. Date: Aug. 30, 2007

(54) Carbohydrase Inhibitors Derived From Chestnut And Use Thereof

(75) Inventors: Takahiro Tsujita, Ehime (JP); Takeshi Takaku, Ehime (JP)

Correspondence Address: FITCH, EVEN, TABIN & FLANNERY, P. O. BOX 18415, WASHINGTON, DC, 20036, US

(73) Assignees: TakahiroTSUJITA, Ehime (JP); Takeshi TAKAKA, Ehime (JP); Chuon Co., Ltd., Ehime (JP)

(21) Appl. No.: 11/662,638

(22) PCT Filed: Sep. 13, 2005
(86) PCT No.: PCT/JP05/17215

§ 371 Date: Mar. 13, 2007

(30) Foreign Application Priority Data
Sep. 13, 2004 (JP) 2004-265281

(51) Int. Cl. A61K 36/49

(52) U.S. Cl. 424/769

(57) ABSTRACT

The present invention provides a plant-derived carbohydrase inhibitor, wherein the inhibitor is effective for preventing or alleviating diabetes, or preventing obesity, and foods, drinks, and medicines containing the same. The present invention is accomplished by use of an α-amylase inhibitor or an α-glucosidase inhibitor as a carbohydrase inhibitor that is extracted from astringent skins of a chestnut using ethanol or aqueous ethanol solution. The present invention can also be accomplished by adding the carbohydrase inhibitor to a food or medical composition as an active ingredient for delaying saccharide digestion or absorption, suppressing a rise in postprandial blood glucose levels or blood insulin levels, or preventing obesity.

### TECHNICAL FIELD

The present invention relates to carbohydrase inhibitors derived from chestnut, and in particular of inhibitors to α-amylase or α-glucosidase. Furthermore, the present invention relates to various uses of the carbohydrase inhibitors, and specifically to medical compositions and food compositions which utilize the physiological actions of the inhibitors.

### BACKGROUND ART

The prevalence of diabetes is increasing due to changes in modern lifestyles. In Japan, it is estimated that the number of diabetic patients, including potential diabetic patients, is more than 15 million. Most of the diabetic patients suffer from type II diabetes. Type II diabetes mellitus is closely related to obesity, and causes chronic hyperglycemia due to insulin resistance, etc. Furthermore, type II diabetes causes complications, such as retinopathy, nephritis, and neurological disorders. Diet and exercise therapy are the key factors for preventing and treating type II diabetes. In dieting, controlling blood glucose levels in everyday life is especially important. Blood glucose levels are greatly affected by the saccharides (starches, glycogen, sugars, etc.) contained in food. These saccharides are decomposed by the actions of α-amylase and α-glucosidase, which are digestive enzymes (carbohydrases). α-Amylase is an endo-type enzyme that hydrolyzes the α-1,4-glucoside linkages of starches and glycogen. These enzymes are contained in the saliva and pancreatic fluid of animals, and transform starches and the like into maltose, etc., in the alimentary canal.

Disaccharides, such as maltose and sucrose, are transformed into glucose by hydrolysis due to α-glucosidase, which exists in the cell membrane of the small intestine mucous membrane, and are absorbed. Maltase, which decomposes maltose, and sucrase, which decomposes sucrose, are typical α-glucosidases. Glucose absorbed from the small intestine is carried into the blood, and raises the blood glucose level. Therefore, to inhibit a superfluous energy supply or control blood glucose levels, in other words, to prevent or treat obesity and diabetes, it is very important to control the activity of these enzymes such as α-amylase and α-glucosidase.

Much research has been conducted on substances that inhibit the action of carbohydrases and many carbohydrase inhibitors have been discovered. Examples include a protein-based substance that is derived from wheat [O'Donnell M D and McGeeney K F.: Purification and properties of an alpha-amylase inhibitor from wheat. Biochim. Biophys. Acta, 422, 159-169 (1976)]; a polysaccharide extracted from soybean (Japanese Unexamined Patent Publication No. 1991-290187; protein-based substances NSA1-I and NSA1-II extracted from cocoyam (Colocasia esculenta) (Japanese Unexamined Patent Publication No. 1991-294300; crude extracts from laurel (Laurus nobillis L) (Japanese Unexamined Patent Publication No. 1992-27389); extracts from guava leaves (Japanese Unexamined Patent Publication No. 1995-59539); banaba (Laqerstroemia speciosa) extracts obtained by using hot water [Hosoyama H, Sugimoto A, Suzuki Y, et al.: Isolation and Quantitative Analysis of the alpha-amylase Inhibitor in Lagerstroemia speciosa (L.) Pers. (Banaba) J. Pharm. Soc. Jpn., 123, 599-605, (2003)]; extracts from the above-the-ground root of ephedra (Ephedra Herb) (Japanese Unexamined Patent Publication No. 1997-2963); extracts from black rice (Japanese Unexamined Patent Publication No. 2004-91462); etc. Moreover, an oligosaccharide produced from actinomyces is known as a carbohydrase inhibitor derived from microorganisms.

Furthermore, examples of well known commercially available medicines (anti-diabetic medicines) having inhibition activities against α-glucosidase or α-amylase include acarbose (Glucobay; manufactured by Bayer Yakuhin, Ltd.)[Jenkins D J, Taylor R H, Nineham R. et al.: “Combined use of guar and acarbose in reduction of postprandial glycaemia”, Lancet 2(8149) 924-927 (1979)] and voglibose (Basen; manufactured by Takeda Pharmaceutical Company Limited)[Yoshio Goto, Shigeaki Baba, Masakazu Nakagawa, et al.: “Effectiveness of AO-128, an α-glucosidase inhibitor, for treating non-insulin dependent diabetes mellitus.” IGAKU NO AYUMI 160 943-971(1992)]. Furthermore, the anti-obesity action of carbohydrase inhibitors is disclosed in a document written by Svensson, et al., [Svensson B, Fukuoka K, Nielsen P K, et al.: “Proteinaceous amylase inhibitors”, Biochim. Biophys. Acta, 1696, 145-156 (2003)] and one written by Udani, et al., [Udani J, Hardy M and Madsen D C: “Blocking carbohydrate absorption and weight loss: A clinical trial using Phase 2 brand proprietary fractionated white bean extract”, Alternative Medicine Review 9, 63-69 (2003)].

As described above, many α-amylase inhibitors and α-glucosidase inhibitors have been proposed. However, in order to put these substances into practical use as effective agents for preventing or treating diabetes or obesity, in addition to the strength of the inhibitory activity against α-amylase or α-glucosidase, studies are necessary from many viewpoints, such as safety and the existence of side effects in the body, and the availability of a stable supply of raw materials, etc. Given this, known inhibitors are not necessarily satisfactory.

### DISCLOSURE OF THE INVENTION

An object of the present invention is to provide a carbohydrase inhibitor obtained from chestnut, and specifically a carbohydrase inhibitor that exhibits an inhibitory activity against α-amylase or α-glucosidase. Another object of the present invention is to provide a carbohydrase inhibitor that is safe to the body and prepared from a material that can be supplied stably.

The carbohydrase inhibitor can delay the digestion and absorption of saccharides from the alimentary canal. This makes it possible to reduce a rise in blood glucose levels after a meal. Furthermore, because the carbohydrase inhibitor can delay the digestion and absorption of saccharides from the alimentary canal, it is expected to have an anti-obesity effect. Therefore, the present invention provides medical compositions and food compositions using these physiological actions of the carbohydrase inhibitor.

In order to find novel carbohydrase inhibitors, the present inventors conducted screening using materials which are generally disposed in daily life, such as juice extraction lees from citrus fruits, juice extraction lees from aojiru (juice from a green, leafy vegetable), peels of various kinds of fruit, bittern, chitin and chitosan of crustacea, and testa or gut of fish. As a result, they found that solvent extracts from the astringent skins of chestnut have a strong α-amylase inhibitory activity or α-glucosidase inhibitory activity. Furthermore, they found that these solvent extracts could reduce a rise in blood glucose level for normal and diabetic rats, and humans after meals (or after eating saccharides). The present invention was accomplished based on these findings. In other words, the present invention includes the following features:
Item 1. A carbohydrase inhibitor comprising chestnut astringent skin extract.
Item 2. The carbohydrase inhibitor according to Item 1, which is obtained by subjecting astringent skin of chestnut to extraction using ethanol or aqueous ethanol solution.
Item 3. The carbohydrase inhibitor according to Item 2, wherein the aqueous ethanol solution contains 5-95 vol. % of ethanol.
Item 4. The carbohydrase inhibitor according to Item 2, wherein the aqueous ethanol solution contains 10-90 vol. % of ethanol.
Item 5. The carbohydrase inhibitor according to Item 2, wherein the aqueous ethanol solution contains 15-90 vol. % of ethanol.
Item 6. The carbohydrase inhibitor according to Item 2, wherein the aqueous ethanol solution contains 20-90 vol. % of ethanol.
Item 7. The carbohydrase inhibitor according to Item 2, wherein the aqueous ethanol solution contains 25-90 vol. % of ethanol.
Item 8. The carbohydrase inhibitor according to Item 2, wherein the aqueous ethanol solution contains 50-90 vol. % of ethanol.
Item 9. The carbohydrase inhibitor according to Item 2, wherein the aqueous ethanol solution contains 50-80 vol. % of ethanol.
Item 10. The carbohydrase inhibitor according to Item 2, wherein the aqueous ethanol solution contains 50-75 vol. % of ethanol.
Item 11. The carbohydrase inhibitor according to any one of Items 1 to 10, wherein the carbohydrase to be inhibited is α-amylase, α-glucosidase, or both.
Item 12. A composition for delaying saccharide digestion and absorption, the composition comprising a carbohydrase inhibitor according to any one of Items 1 to 11 as an active ingredient.
Item 13. A composition for reducing a postprandial rise in blood glucose levels comprising, as an active ingredient, a carbohydrase inhibitor of any one of Items 1 to 11.
Item 14. A composition for ameliorating hyperglycemia comprising, as an active ingredient, a carbohydrase inhibitor of any one of Items 1 to 11.
Item 15. An anti-obesity composition comprising, as an active ingredient, a carbohydrase inhibitor of any one of Items 1 to 11.
Item 16. A food composition comprising a carbohydrase inhibitor of any one of Items 1 to 11.
Item 17. The food composition according to Item 16, which is a beverage or a food high in carbohydrates, such as a noodle, a bread or confectioneries.
Item 18. A food composition comprising an effective amount of a carbohydrase inhibitor according to any one of Items 1 to 11 as an active ingredient for delaying saccharide digestion and absorption.
Item 19. The food composition according to Item 18, which has the property of delaying saccharide digestion and absorption, and whose package has a note stating that the food composition is suitable for delaying digestion and absorption of saccharides.
Item 20. A food composition comprising an effective amount of a carbohydrase inhibitor according to any one of Items 1 to 11 as an active ingredient for reducing a postprandial rise in blood glucose or blood insulin levels, or for ameliorating hyperglycemia.
Item 21. The food composition according to Item 20, which has the property of suppressing postprandial rise in blood glucose levels or for ameliorating hyperglycemia, and whose package has a note that the food composition is suitable for suppressing a postprandial rise in blood glucose levels or for ameliorating hyperglycemia.
Item 22. A food composition comprising an effective amount of carbohydrase inhibitor according to any one of Items 1 to 11 as an active ingredient for preventing obesity.
Item 23. The food composition according to Item 21, which has an anti-obesity effect, and whose package has a note stating that the food composition is suitable for preventing obesity.
Item 24. A medical composition comprising, as an active ingredient, a carbohydrase inhibitor according to any one of Items 1 to 11.
Item 25. The medical composition according to Item 24, which is a medicine for preventing or treating diabetes.
Item 26. The medical composition according to Item 24, which is an anti-obesity medicine.
Item 27. A method for suppressing a postprandial rise in blood glucose levels or ameliorating hyperglycemia of a subject, comprising injecting or otherwise administering a carbohydrase inhibitor according to any one of Items 1 to 11 to the subject.
Item 28. A method for preventing or reducing obesity comprising injecting or otherwise administering a carbohydrase inhibitor according to any one of Items 1 to 11 to the subject.
Item 29. Use of a carbohydrase inhibitor according to any one of Items 1 to 11, for producing a composition for delaying saccharide digestion and absorption.
Item 30. Use of a carbohydrase inhibitor according to any one of Items 1 to 11, for producing a composition for suppressing a postprandial rise in blood glucose levels.
Item 31. Use of a carbohydrase inhibitor according to any one of Items 1 to 11, for producing a composition for ameliorating hyperglycemia.
Item 32. Use of a carbohydrase inhibitor according to any one of Items 1 to 11, for producing an anti-obesity composition.

### BRIEF DESCRIPTION OF DRAWINGS

FIG. 1 shows the effect of 100 vol. % ethanol extract (-Δ-), 75 vol. % aqueous ethanol extract (-▪-), 50 vol. % aqueous ethanol extract (-□-), 25 vol. % aqueous ethanol extract (-●-), and water extract (-◯-) of chestnut astringent skin on α-amylase activity (%) (Experiment 1).

FIG. 2 shows the effect of 100 vol. % ethanol extract (-Δ-), 75 vol. % aqueous ethanol extract (-▪-), 50 vol. % aqueous ethanol extract (-□-), 25 vol. % aqueous ethanol extract (-●-), and water extract (-◯-) of chestnut astringent skin on α-glucosidase (maltase) activity (%) (Experiment 2(2)).

FIG. 3 shows the effect of 100 vol. % ethanol extract (-Δ-), 75 vol. % aqueous ethanol extract (-▪-), 50 vol. % aqueous ethanol extract (-□-), 25 vol. % aqueous ethanol extract (-●-), and water extract (-◯-) of chestnut astringent skin on α-glucosidase (sucrase) activity (%) (Experiment 2(3)).

FIG. 4 shows the change in the blood glucose levels (mg/dl) after administering 75 vol. % aqueous ethanol extract of chestnut astringent skin (300 mg/kg of body weight) and starch (2 g/kg body weight) to normal rats (-●-) (Experiment 3). As a control, the change in the blood glucose level of normal rats that was administered with only starch (2 g/kg of body weight) is also shown (-◯-).

FIG. 5 shows the change in the blood insulin levels (ng/ml) after administering 75 vol. % aqueous ethanol extract of chestnut astringent skin (300 mg/kg of body weight) and starch (2 g/kg body weight) to normal rats (-●-) (Experiment 3). As a control, the change in the blood insulin level of normal rats that was administered with only starch (2 g/kg of body weight) is also shown (-◯-).

### BEST MODE FOR CARRYING OUT THE INVENTION

(1) Carbohydrase Inhibitor

A carbohydrase inhibitor of the present invention can be obtained by extracting from astringent skin of a chestnut (Castanea crenata) belonging to the Castanea genus with ethanol or aqueous ethanol solution.

The astringent skin of a chestnut include tannins, gallic acid, flavonoids, etc., and is believed to be effective in treating heat rash and skin burn because of an antiphlogistic effect. It is also believed to be effective in preventing lifestyle-related diseases, such as arteriosclerosis, by improving blood circulation and preventing deposition of cholesterol in the blood vessels. However, there is no report stating that it inhibits the actions of carbohydrases, such as α-amylase and α-glucosidase, nor that it suppresses a rise in blood glucose levels by means of this inhibitory effect.

The form of the chestnut astringent skin to be subjected to extraction is not limited and may be raw, dried or soaked with water, and the chestnut astringent skin may be crushed or pulverized into a desired shape such as chips or powder.

The solvent used for extraction is water, organic solvents or a mixture thereof can be used. Preferable organic solvent is ethanol.

When a mixture of water and ethanol (aqueous ethanol solution) is used as an extractant, the content of ethanol is not limited, and is for example usually 5-95 vol. %, preferably 10-90 vol. %, more preferably 15-90 vol. %, still more preferably 20-90 vol. %, still yet more preferably 25-90 vol. %, further more preferably 50-90 vol. %, and particularly preferably 50-80 vol. %, and particularly more preferably 50-75 vol. %.

Generally used methods can be employed in the extraction method. There is no restriction on the extraction method, and examples thereof include a method wherein astringent skin of a chestnut (raw, dried, crushed, or pulverized), is subjected to cold extraction, hot extraction, or dipped in a solvent while heating; a percolation method, etc. The extraction temperature is not particularly limited and can be suitably selected from the range of 4 to 100° C., and preferably 25 to 60° C. Dipping of the chestnut astringent skin may be conducted while the solvent is allowed to stand, or while stirring or shaking. The extraction time is not particularly limited and can be suitably selected from the range of 1 hour to 2 weeks. The volume of the extractant is also not limited. It is preferable that extraction be repeated 2 to 3 times using a solvent in an amount 10 to 30 times (weight ratio) that of the chestnut astringent skin being extracted on a dry-weight basis.

It is also possible to conduct extraction using a solvent in a supercritical or subcritical state (supercritical extraction method or subcritical extraction method). In supercritical and subcritical extraction methods, extraction is conducted using a solvent in a supercritical or subcritical state (in a state that both the temperature and pressure of the solvent exceed critical values, in other words, the solvent is in an intermediate state between liquid and gas). Examples of extractants are carbon dioxide, ethylene, ethane, propane, water, etc.; however, from the viewpoint of safety and non-toxicity, etc., carbon dioxide is preferable.

The extraction pressure and temperature are not limited as long as the extractant becomes supercritical or subcritical, and can be suitably selected depending on the extractant to be used. Specifically, the extraction pressure can be selected from a range of 3-70 MPa, and, for example, when carbon dioxide is used as an extractant, it is preferable to select the extraction pressure from a range of 5-40 MPa. The extraction temperature can usually be selected from the range of 25-200° C., and preferably 25-100° C. There is no restriction; however, it is possible to use an entrainer to enhance the solubility of the extractant. Examples of entrainers include water; methanol, ethanol, and like C₁-C₄ lower alcohols; acetone, acetonitrile, etc. When an entrainer is used, it is usually preferable that the content of the entrainer in the extractant be 0.00001-50.0 wt %, and more preferably 0.0001-10 wt %. The extraction time is not limited and can be suitably selected from the range of 2 hours to 2 weeks.

If necessary, solid substances are removed from the obtained extract by filtration, centrifugation and/or other solid-liquid separation methods. Depending on the mode of use, the extract may be used as is, or partially concentrated or dried by evaporating the solvent, and the extract can be used as a chestnut essence or dried chestnut essence. The thus obtained chestnut astringent skin extract, preferably the extract obtained by subjecting astringent skin of chestnut to extraction using ethanol or aqueous ethanol solution, has an inhibitory activity against carbohydrases, such as α-amylase and α-glucosidase as described in the Examples below. Therefore, the chestnut astringent skin extract can be used for foods, medicines, feeds, reagents, etc., as an ingredient, for inhibiting the activities of α-amylase, α-glucosidase and like carbohydrases.

The above-described chestnut essence or dried chestnut essence may be purified by washing it with a solvent in which the chestnut essence is insoluble. It is also possible to use the chestnut essence or dried chestnut essence by dissolving or suspending it into an additional suitable solvent.

The above-described chestnut essence or dried chestnut essence may be highly purified using a known purifying method, and the thus obtained purified substance may be used as a carbohydrase inhibitor. There is no restriction on the purification method, and an example thereof is a countercurrent distribution method, chromatography, etc., wherein carbohydrase inhibition activities (e.g., α-amylase inhibitory activity and α-glucosidase inhibitory activity) are measured and a fraction having at least one of these activities is selected. Several methods for measuring α-amylase inhibitory activity and α-glucosidase inhibitory activity are known, and any such method can be employed. Specifically, purification can be conducted by following the method as described later in the Examples. The purified extract obtained by employing any of various purification methods may be dried by decompression drying, freeze-drying or the like standard drying method and can be used as a carbohydrase inhibitor.

(2) Use of Carbohydrase Inhibitor

The above-described carbohydrase inhibitor of the present invention can be used as a reagent (chemical product) as an α-amylase inhibitor or α-glucosidase inhibitor due to its α-amylase inhibitory activity or α-glucosidase inhibitory activity.

A carbohydrase inhibitor of the present invention has the property of delaying digestion and absorption of saccharides in vivo (intestinal) and suppressing a postprandial rise of blood glucose levels (hyperglycemia) due to its α-amylase inhibitory activity or α-glucosidase inhibitory activity. Therefore, the carbohydrase inhibitor of the present invention can be used as an active ingredient of a composition for delaying the digestion and absorption of saccharides (a digestion and absorption retardant), a composition for suppressing a postprandial rise in blood glucose levels (an inhibitor of rising blood glucose levels), or a composition for ameliorating hyperglycemia (hyperglycemia improver).

(3) A Composition for Delaying Digestion and Absorption of Saccharides, a Composition for Suppressing a Postprandial Rise in Blood Glucose Levels, a Composition for Ameliorating Hyperglycemia, and an Anti-Diabetic Composition.

As described above, the present invention provides a composition for delaying digestion and absorption of saccharides, a composition for suppressing a postprandial rise in blood glucose levels, and a composition for ameliorating hyperglycemia.

The composition for delaying the digestion and absorption of saccharides may contain the above-described carbohydrase inhibitor in an amount that is effective for delaying the digestion and absorption of saccharides in the alimentary canal. A composition for suppressing a postprandial rise in blood glucose levels or the composition for ameliorating hyperglycemia may contain the above-described carbohydrase inhibitor in an amount that is effective for suppressing the postprandial rise in blood glucose levels. Usually, a composition for delaying the digestion and absorption of saccharides, composition for suppressing a postprandial rise in blood glucose levels or a composition for ameliorating hyperglycemia should contain 0.1-100 parts by weight of carbohydrase inhibitor of the present invention per 100 parts by weight of the total composition. A composition for delaying the digestion and absorption of saccharides, a composition for suppressing a postprandial rise in the blood glucose level, or a composition for ameliorating hyperglycemia may comprise, in addition to the above-described carbohydrase inhibitor, pharmaceutically acceptable carriers and additives and/or carriers and additives that are permitted to be added to foods.

The carbohydrase inhibitor of the present invention can be used as an active ingredient of an anti-obesity composition (anti-obesity agent) that prevents obesity caused by hyperphagia, since the carbohydrase inhibitor has the property of inhibiting digestion of saccharides, such as starches and sugars contained in foods, and prevents them from being absorbed as energy. Therefore, the present invention provides an anti-obesity composition comprising the carbohydrase inhibitor as an active ingredient. The composition may contain the carbohydrase inhibitor in an amount that is effective for resolving or suppressing obesity. Usually, the anti-obesity composition contains 0.1-100 parts by weight of the carbohydrase inhibitor of the present invention per 100 parts by weight of the total composition. The anti-obesity composition may comprise, in addition to the above-described carbohydrase inhibitor, pharmaceutically acceptable carriers and/or additives or carriers and additives that are permitted to be added to foods.

(4) Food Composition and Medical Composition

As an example of a more specific and practical mode, the carbohydrase inhibitor of the present invention can be used as an active ingredient of a food or medical composition, and prepared into food or medicine. Due to its α-amylase inhibitory activity or α-glucosidase inhibitory activity, such a food or medical composition of the present invention has the property of delaying digestion and absorption of saccharides in vivo (intestinal), suppressing a postprandial rise of blood glucose levels, ameliorating hyperglycemia, and/or preventing obesity.

Therefore, the present invention provides a food or medical composition that has the above-described effects by comprising the carbohydrase inhibitor. Such food or medical compositions are not limited to only those for humans but also include those for various animals, in particular, other mammals. Therefore, the food compositions include foods for animals such as cats, dogs, and the like pets, and the medical compositions include those for animals other than humans.

(4-1) Food Composition

Since the food composition of the present invention has a property of delaying the digestion and absorption of saccharides, suppressing a rise in postprandial blood glucose levels, and/or ameliorating hyperglycemia as described above, the food composition of the present invention has an effect for preventing diabetes and/or the progress thereof, or preventing diseases caused by postprandial hyperglycemia. Therefore, the food composition of the present invention is useful as a health food or a functional food for a subject (including human subjects) suffering from a relatively high blood glucose level or a subject (including human subjects) whose blood glucose level may be of concern. Such a food composition is not limited as long as it comprises the carbohydrase inhibitor in an amount effective for delaying the digestion and absorption of saccharides in the alimentary canal, suppressing a rise in postprandial blood glucose levels, or ameliorating hyperglycemia. If necessary, the composition may contain carriers and/or other additives permitted to be added to foods.

Since the food composition of the present invention has an effect for delaying the digestion and absorption of saccharides in vivo (intestinal), it is possible to provide a food composition of the present invention as a so-called anti-obesity food, i.e., the subject will not easily gain weight through eating the food. The food composition is not limited as long as it comprises the carbohydrase inhibitor in an amount effective for delaying the digestion and absorption of saccharides in the alimentary canal, and, if necessary, the composition may contain carriers and/or other additives permitted to be added to foods.

The forms of such food compositions are not limited, and the carbohydrase inhibitor may be prepared into supplements (functional foods) having the form of tablets, pills, capsules, granules, pulvis, powders, troches, solutions (drinks), etc., together with carriers and/or other additives permitted to be added to foods, if necessary.

The food compositions of the present invention include foods (for example, foods for specified health use, dietary supplements, functional foods, etc.) that have various effects due to their α-amylase inhibitory activity or α-glucosidase inhibitory activity by comprising the carbohydrase inhibitor. The foods for specified health use encompassed by the present invention include the foods which have a property of, by containing the carbohydrase inhibitor, delaying the digestion and absorption of saccharides, suppressing a rise in postprandial blood glucose levels, and/or ameliorating hyperglycemia, and therefore such foods have labels on their packages stating that they are useful for delaying digestion and absorption of saccharides, suppressing a rise in postprandial blood glucose levels (hyperglycemia), and/or ameliorating hyperglycemia. There is no specific limitation on the expressions of the labels and include, for example, “suitable for those who care about blood glucose levels”, “suitable for those who have relatively high blood glucose level”, or “moderating absorption of saccharides”.

The foods for specified health use encompassed by the present invention include those comprising the carbohydrase inhibitor and having an effect for delaying digestion and absorption of saccharides, and therefore such foods have labels on their packages stating that they are useful for reducing or preventing obesity (i.e., loosing weight). The expressions on the package is not limited, and examples thereof include “suitable for those who are concerned about their weight”, “suitable for those who are over-weight”, etc.

Examples of such foods include milk-based beverages, lactobacillus beverages, fruit juice-containing beverages, soft drinks, carbonated beverages, fruit juices, vegetable juices, vegetable and fruit juice-containing beverages, alcoholic beverages, powdered beverages, coffee, black tea, green tea, barley tea, and like beverages; custard puddings, milk puddings, souffle puddings, fruit juice-containing puddings and like puddings; jelly, bavarois, yogurt, and the like desserts; ice cream, iced milks, lact-ice, milk ice-cream, fruit juice-containing ice cream, soft ice cream, popsicles, sorbet, iced confectionery, and like cold sweets; chewing gum, bubble gum, and like gums (stick gums, sugar-coated grain gums, etc.); chocolates, such as marble-chocolate and like coated chocolates, as well as strawberry chocolate, blueberry chocolate, melon chocolate and like flavor-added chocolates; hard candies (including bonbons, butterballs, marbles, etc.), soft candies (including caramel, nougat, gummy candy, marshmallows, etc.), drops, toffee, and like caramels; cakes, hard biscuits, cookies, okaki (rice cracker), senbei (rice cracker), and like baked confections (these are referred to as confectioneries); breads; consomme soups, potages and like soups; miso, soy sauce, dressings, ketchup, sauces, furikake (seasoned powder for sprinkling over rice) and like seasonings; strawberry jam, blueberry jam, marmalade, apple jam, apricot jam, preserves and like jams; red wine and like fruit-based liquors; compote of cherries, apricots, apples, strawberries, peaches and like processed fruits; hams, sausages, roast pork and like processed meats; fish ham, fish sausage, fish surimi (pasted fish meat), kamaboko (pureed fish loaf), chikuwa (pureed and steamed fish cake), hanpen (pounded fish cake), satsumaage (deep fried fish paste), datemaki (sweet omelet with fish past), whale-bacon and like “fish” cakes; udon (thick white wheat noodles), hiyamugi (thin udon), somen (fine white wheat noodles), buckwheat noodles, Chinese noodles, spaghetti, macaroni, bifun (rice vermicelli), harusame (thin potato starch noodles), wonton and like noodles; and like various processed foods. Preferable examples are beverages, and foods high in carbohydrates, such as noodles, breads, and confectioneries.

The amount of the carbohydrase inhibitor contained in the above-mentioned food composition and the amount of the carbohydrase inhibitor intaken are not limited, and can be suitably selected from a wide range depending on the kind of food compositions, targeted functions and effects, and other conditions. The amount intaken thereof varies depending on the types of the food composition; however, the amount of carbohydrase inhibitor (for example, based on the dry weight of astringent skin of Castanea on a dry weight) taken per instance by a person whose body weight is 60 kg can be suitably selected form the range of about 10 to 200,000 mg/(60 kg body weight).

(4-2) Medical Composition

The medical composition of the present invention comprising a carbohydrase inhibitor as an active ingredient can be effectively used as an anti-diabetic medicine due to its effect of suppressing a postprandial rise in blood glucose levels (hyperglycemia) by delaying digestion and absorption of saccharides in vivo (intestinal).

The anti-diabetic medicine broadly encompasses those that can prevent or improve diabetes. Specifically, the anti-diabetic medicine of the present invention includes those that can prevent an onset of diabetes in a subject (including humans and other animals) who has the potential of suffering from an onset of diabetes, due to its effect for suppressing a postprandial rise in blood glucose levels. Furthermore, the anti-diabetic medicine of the present invention encompasses those that have an effect for ameliorating the hyperglycemic condition of a subject (including humans and other animals). The anti-diabetic medicine of the present invention also encompasses those that have an effect for preventing or ameliorating diseases attributable to hyperglycemia, such as diabetic complications, by suppressing or ameliorating blood glucose levels (reducing the blood glucose level from hyperglycemic condition). Note that the diabetes targeted by the present invention is preferably insulin-independent type II diabetes.

Diabetic complications are general or local diseases directly or indirectly caused by diabetes. Specific examples thereof are diabetic acidosis, diabetic xanthoma, diabetic myatrophy, diabetic ketosis, diabetic coma, diabetic stomach disorders, diabetic gangrene, diabetic ulcer, diabetic diarrhea, diabetic microangiopathy, diabetic uterosclerosis, diabetic cardiomyopathy, diabetic neuropathy, diabetic nephropathy, diabetic blister, diabetic cataract, diabetic dermatitis, diabetic scleredema, diabetic retinopathy, diabetic necrobiosis lipoidica, diabetic blood flow obstructions, etc.

The above-described carbohydrase inhibitor can be used as an anti-diabetic medicine (medical composition) as is. However, it is preferable that the carbohydrase inhibitor be used as an anti-diabetic medicine (medical composition) comprising the carbohydrase inhibitor in an amount effective for suppressing a rise in blood glucose levels together with pharmaceutically acceptable carriers and/or additives.

The medical composition of the present invention comprising a carbohydrase inhibitor as an active ingredient can be effectively used as an anti-obesity medicine due to its effect for delaying digestion and absorption of saccharides in vivo (intestinal). The above-described carbohydrase inhibitor can be used as an anti-obesity medicine (medical composition) by itself; however, it preferable that the carbohydrase inhibitor be used as an anti-obesity medicine (medical composition) comprising an effective amount of carbohydrase inhibitor for preventing obesity, and pharmaceutically acceptable carriers and/or additives.

The form of administration of the medical composition (form of a pharmaceutical preparation) can be suitably selected depending on the administration route. Medical compositions are generally classified into the following groups: orally administered medicines, nasally administered medicines, vaginally administered medicines, suppositories, sublingual tablets, non-orally administered medicines (injection or drops), etc. In the present invention, it is preferable that the composition be administered orally. By following a standard method, the composition of the present invention can be formed, or prepared into solid pharmaceutical preparations, such as tablets, pills, pulvis, powders, granules, troches, capsules, etc.; or liquid pharmaceutical preparations, such as solutions, suspensions, emulsions, syrups, elixirs, etc.

In manufacturing these pharmaceutical preparations, depending on the form of administration, it is possible to use general carriers such as excipients, diluents, binders, moisturizing agents, disintegrators, disintegration inhibitors, absorption accelerators, lubricants, solubilizers, buffers, emulsifiers, suspension agents, etc. Examples of additives include those that usually used depending on the form of administration, such as stabilizer, preservative, buffer, isotonizing agent, chelating agent, pH controller, surfactant, coloring agent, fragrances, flavoring agent, sweetening agent, etc.

The amount of carbohydrase inhibitor contained in the medical composition of the present invention depends on the form of the pharmaceutical preparation and cannot be defined unconditionally, but is usually selected from a range so that the final pharmaceutical preparation contains carbohydrase inhibitor in an amount of 0.001-100 wt %, and preferably 0.01-80 wt %.

The administration amount of the medical composition are not limited, and can be suitably selected from a wide range depending on the targeted treatment effects, administration method, treatment period, sex and or age of the subject, etc. The administration amount, e.g., the dose of carbohydrase inhibitor administered to a person whose body weight is 60 kg, depends on the administration route, and can be suitably selected from a range of about 10 to 200,000 mg/(60 kg body weight).

### EXAMPLES

The following Examples and Experiments are intended to illustrate the present invention in further detail, and not to limit the scope of the invention. In the Examples and Experiments, “%” means “% w/w” unless otherwise specified.

### Preparation Example 1

### Chestnut Astringent Skin Extract (Ethanol Concentration in Extractant: 100 vol. %)

One hundred milliliters of 100 vol. % ethanol was added to 50 g of the chestnut astringent skin, followed by stirring at 35° C. for 15 hours. The mixture was then centrifuged at 3000 g for 15 minutes, and the resulting supernatant was concentrated in a rotary evaporator and lyophilized, to thereby obtain 1.59 g of an ethanol extract of chestnut astringent skin (lyophilized product).

### Preparation Example 2

### Chestnut Astringent Skin Extract (Ethanol Concentration in Extractant: 75 vol. %)

Using 75 vol. % aqueous ethanol solution but not 100 vol. % ethanol as an extractant, 50 g of the chestnut astringent skin was subjected to the same procedure as in Preparation Example 1, to thereby obtain 2.45 g of an aqueous ethanol extract of chestnut astringent skin (lyophilized product).

### Preparation Example 3

### Chestnut Astringent Skin Extract (Ethanol Concentration in Extractant: 50 vol. %)

Using 50 vol. % aqueous ethanol solution but not 100 vol. % ethanol as an extractant, 50 g of the chestnut astringent skin was subjected to the same procedure as in Preparation Example 1, to thereby obtain 2.70 g of an aqueous ethanol extract of chestnut astringent skin (lyophilized product).

### Preparation Example 4

### Chestnut Astringent Skin Extract (Ethanol Concentration in Extractant: 25 vol. %)

Using 25 vol. % aqueous ethanol solution but not 100 vol. % ethanol as an extractant, 50 g of the chestnut astringent skin was subjected to the same procedure as in Preparation Example 1, to thereby obtain 2.59 g of an aqueous ethanol extract of chestnut astringent skin (lyophilized product).

### Preparation Example 5

### Chestnut Astringent Skin Extract (Ethanol Concentration in Extractant: 0 vol. %)

Using Water but not 100 vol. % ethanol as an extractant, 50 g of the chestnut astringent skin was subjected to the same procedure as in Preparation Example 1, to thereby obtain 1.85 g of an aqueous extract of chestnut astringent skin (lyophilized product).

### Comparative Preparation Example

### Hot Water Extract of Guava Leaves

Dried guava leaves were pulverized to prepare a guava leaf powder. Two liters of water was added to 100 g of the guava leaf powder, followed by stirring at 100° C. for 1 hour. The mixture was then centrifuged at 3000 g for 15 minutes, and the resulting supernatant was concentrated in a rotary evaporator and lyophilized, to thereby obtain 17.6 g of a hot water extract of guava leaves.

### Experiment 1

### α-Amylase Inhibitory Activity Test

(1) The chestnut astringent skin extracts of Preparation Examples 1 to 5 and hot water extract of guava leaves (Comparative Preparation Example) were tested for inhibitory activity against α-amylase. Each of the above extracts was dissolved in 200 mM phosphate buffer (pH 7.0) to final concentrations of 2.7, 5.5, 22, 55 and 110 μg/ml to use as test inhibitors, in this test.

Specifically, 1.0 ml of buffer (200 mM phosphate buffer, pH 7.0), 0.5 ml of 1% aqueous sodium chloride solution, 2.5 ml of 0.25% soluble starch solution in 200 mM phosphate buffer at pH 7.0, and 0.5 ml of one of the test inhibitors at one of the above concentrations were mixed together, and pig pancreatic α-amylase (Sigma) was added in an amount of 50 μl (about 1.6 U; 1 U being the amount required to release 1 mg of maltose from starch per 3 minutes at 20° C. and pH 6.8), followed by a reaction at 37° C. for 30 minutes. Subsequently, 0.5 ml of 8% aqueous sodium hydroxide was added to the reaction mixture to terminate the reaction, and 0.5 ml dinitrosalicylic acid reagent [prepared by mixing 50 ml of potassium sodium tartrate solution (30 g/50 ml of purified water) and 20 ml of 3,5-dinitrosalicylic acid solution (1 g/20 ml of 8% aqueous sodium hydroxide) and diluting the mixture with purified water to 100 ml] was added. The resulting mixture was heated at 100° C. for 5 minutes and then cooled, and its absorbance at 540 nm was measured. The measured absorbance is referred to as B. As a blank test, the above procedure was repeated using 50 μl of purified water in place of 50 μl pig pancreatic α-amylase, and the absorbance at 540 nm was measured. This measured absorbance is referred to as D. Further, the above procedure was repeated using 0.5 ml of purified water in place of 0.5 ml of the test inhibitor, and the absorbance at 540 nm was measured. This measured absorbance is referred to as A. Furthermore, the above procedure was repeated using 0.55 ml of purified water in place of 0.5 ml of the test inhibitor and 50 μl of α-amylase, and the absorbance at 540 nm was measured. This measured absorbance is referred to as C.

The α-amylase activity (%) in each reaction was calculated from the absorbances A, B, C and D by the following equation:
α-Amylase activity(%)={(B−D)/(A−C)□×100

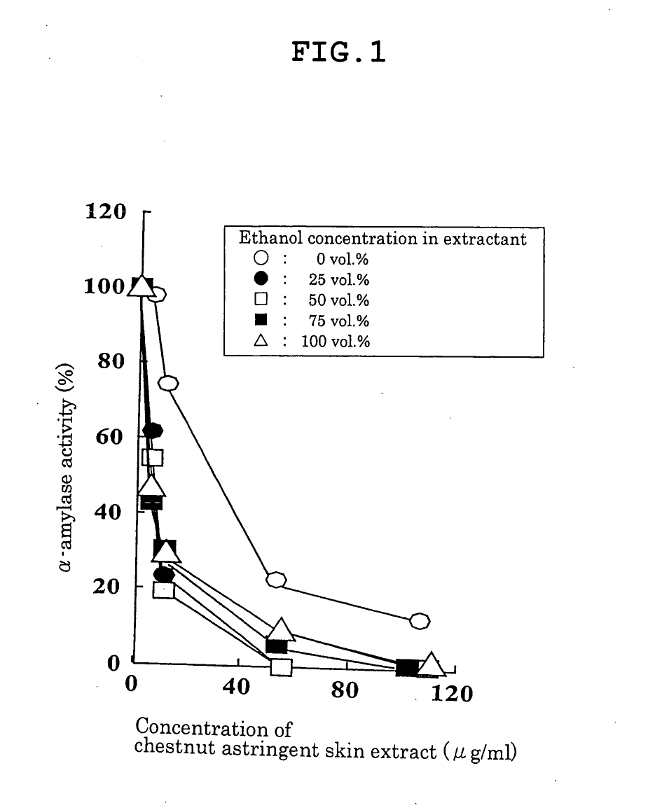

FIG. 1 shows the α-amylase activity (%) of each reaction system, plotting the concentration (μg/ml) of each extract used as a test inhibitor as abscissa. FIG. 1 reveals that all of the chestnut astringent skin extract (100 vol. % ethanol) (-Δ-), chestnut astringent skin extract (75 vol. % aqueous ethanol) (-▪-), chestnut astringent skin extract (50 vol. % aqueous ethanol) (-□-), chestnut astringent skin extract (25 vol. % aqueous ethanol) (-●-) and chestnut astringent skin extract (water) (-◯-) inhibited α-amylase activity in a concentration dependent manner, demonstrating that these extracts have α-amylase inhibitory activity.

The 50% inhibitory concentrations of these extracts were calculated from the inhibitory activities. Table 1 shows the results and the 50% inhibitory concentrations of the chestnut astringent skin extracts (Preparation Examples 1 to 5) and hot water extract of guava leaves (Comparative Preparation Example) measured above in (1). The hot water extract of guava leaves is known to have α-amylase inhibitory activity (e.g., from Japanese Unexamined Patent Publication No. 1995-59539).

TABLE 1
Fifty percentage inhibitory concentrations of the extracts
for α-amylase

| Prep. | | 50% Inhibitory |
|---|---|---|
| Ex. | Chestnut astringent skin extract | concentration |
| 1 | 100 vol. % ethanol | 5.1 μg/ml |
| 2 | 75 vol. % aqueous ethanol | 4.4 μg/ml |
| 3 | 50 vol. % aqueous ethanol | 6.5 μg/ml |
| 4 | 25 vol. % aqueous ethanol | 7.3 μg/ml |
| 5 | water | 21.0 μg/ml |
| Comp. | Guava leaf extract | 25.0 μg/ml |

As is apparent from these results, among these chestnut astringent skin extracts have a low 50% inhibitory concentration against α-amylase of about one-third to about one-fifth of that of the hot water extract of guava leaves, demonstrating that they have extremely high α-amylase inhibitory activity.

### Experiment 2

### α-Glucosidase Inhibitory Activity Test

The chestnut astringent skin extracts (Preparation Examples 1 to 5) and hot water extract of guava leaves (Comparative Preparation Example) were tested for inhibitory activity against α-glucosidases (maltase and sucrase). Each of the above extracts was dissolved in 80 mM phosphate buffer (pH 7.0) to final concentrations of 0.13, 0.25, 0.5 and 1.0 mg/ml to use as test inhibitors in this test.

(1) Preparation of α-Glucosidase Solution

An α-glucosidase solution was prepared according to Anal. Biochem 7: 18-25, 1964. Specifically, small intestines were excised from rats, washed with physiological saline, and everted. The jejunal mucosal cells were scraped with a glass slide, placed in a Teflon® homogenizer containing 80 mM phosphate buffer (pH 7.0) and homogenized on ice. The phosphate buffer was used in an amount of 40 ml with respect to jejunal mucosal cells obtained from 4 rats. The homogenized cells were centrifuged (1000 g, 10 min, 4° C.), and the supernatant was used as an α-glucosidase solution.

(2) Measurement of Maltase Inhibitory Activity

(2-1) Fifty microliters of α-glucosidase solution prepared above in (1) was added to a mixture of 400 μl of 50 mM maltose solution (substrate solution) in phosphate buffer and 50 μl of one of the test inhibitors, and the resulting mixture was maintained at 37° C. for 30 minutes. Subsequently, the reaction was terminated in a boiling water bath for 2 minutes, and the reaction mixture was then ice-cooled. The glucose released in the reaction mixture was measured by a glucose measurement kit (Glucose C-II Test Wako, Wako Pure Chemical Ind. Ltd.). The measured amount of glucose is referred to as B. As a blank test, the above procedure was repeated using 50 μl of purified water in place of 50 μl of the α-glucosidase solution, and the amount of released glucose was measured. This measured amount of glucose is referred to as D. Further, the above procedure was repeated using 50 μl of purified water in place of 50 μl of the test inhibitor, and the amount of released glucose was measured. This measured amount of glucose is referred to as A. Furthermore, the above procedure was repeated using 100 μl of purified water in place of 50 μl of the test inhibitor and 50 μl of the α-glucosidase solution, and the amount of released glucose was measured. This measured amount of glucose is referred to as C.

The maltase activity (%) in each reaction system was calculated from the glucose amounts A, B, C and D by the following equation:
Maltase activity(%)={(B−D)/(A−C)}×100

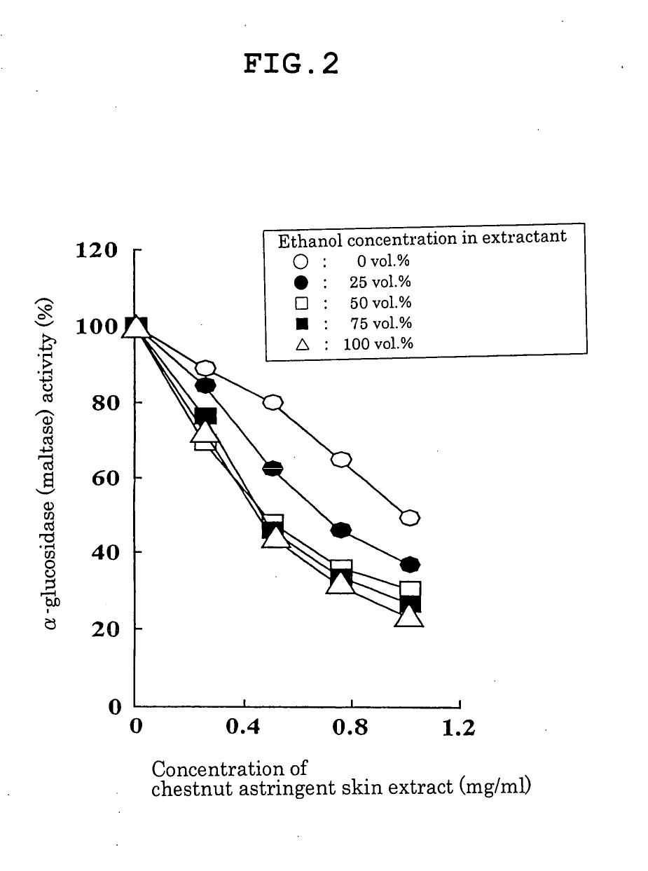

(2-2) FIG. 2 shows the maltase activity (%) of each reaction, plotting the concentration (mg/ml) of each extract used as a test inhibitor [chestnut astringent skin extracts (Preparation Examples 1 to 5)] as abscissa. FIG. 2 reveals that all of the chestnut astringent skin extract (100 vol. % ethanol) (-Δ-), chestnut astringent skin extract (75 vol. % aqueous ethanol) (-▪-), chestnut astringent skin extract (50 vol. % aqueous ethanol) (-□-), chestnut astringent skin extract (25 vol. % aqueous ethanol) (-●-) and chestnut astringent skin extract (water) (-◯-) inhibited maltose activity in a concentration dependent manner, demonstrating that these extracts have α-glucosidase (maltase) inhibitory activity.

(2-3) Table 2 shows the 50% inhibitory concentrations (mg/ml) of the chestnut astringent skin extracts (Preparation Examples 1 to 5) and hot water extract of guava leaves (Comparative Preparation Example) against maltase activity. The hot water extract of guava leaves is known to have α-glucosidase (maltase) inhibitory activity (e.g., “Food Science & Business”, offprint from Nikkei Biotechnology & Business, pp. 108-111, 2003).

TABLE 2
Fifty percentage inhibitory concentrations of the extracts
for maltase

| Prep. | | 50% Inhibitory |
|---|---|---|
| Ex. | Chestnut astringent skin extract | concentration |
| 1 | 100 vol. % ethanol | 0.44 mg/ml |
| 2 | 75 vol. % aqueous ethanol | 0.46 mg/ml |
| 3 | 50 vol. % aqueous ethanol | 0.47 mg/ml |
| 4 | 25 vol. % aqueous ethanol | 0.67 mg/ml |
| 5 | water | 0.99 mg/ml |
| Comp. | Guava leaf extract | 0.56 mg/ml |

Table 2 reveals that, like the chestnut astringent skin extracts showed α-glucosidase (maltase) inhibitory activity equivalent to or better than that of the hot water extract of guava leaves.

(3) Measurement of Sucrase Inhibitory Activity

(3-1) The procedure described above in (2-1) was repeated using, as a substrate solution, 400 μl of 50 mM sucrose solution in phosphate buffer in place of 400 μl of 50 mM maltose solution in phosphate buffer, and the amount of released glucose in the reaction mixture was measured.

The sucrase activity (%) in each reaction was calculated from the glucose amounts A, B, C and D by the following equation:
Sucrase(%)={(B−D)/(A−C)}×100

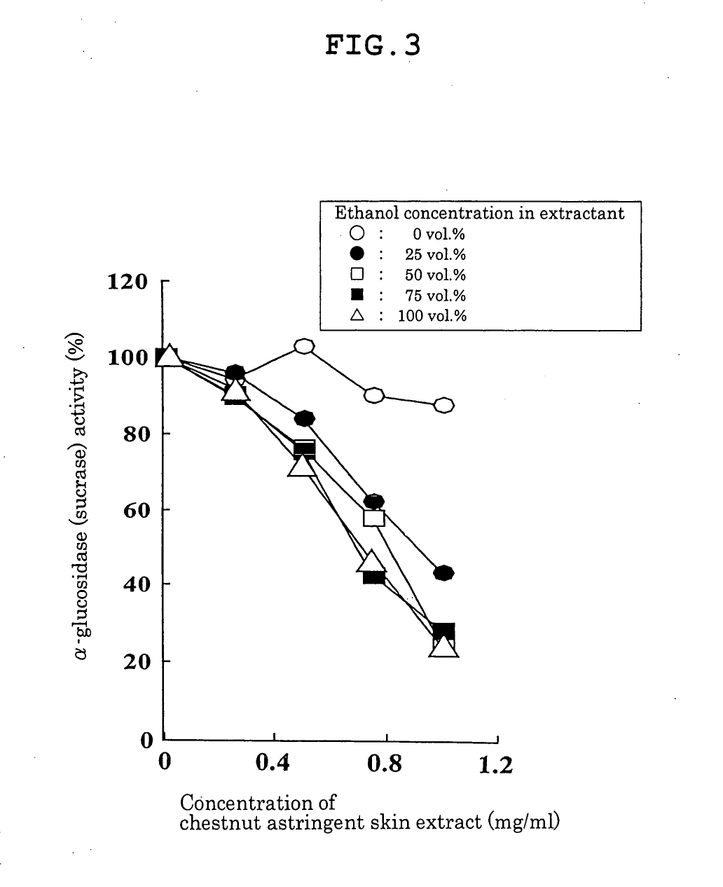

(3-2) FIG. 3 shows the sucrase activity (%) of each reaction, plotting the concentration of each extract used as a test inhibitor [the chestnut astringent skin extracts (Preparation Examples 1 to 5) as abscissa. FIG. 3 reveals that the chestnut astringent skin extract (100 vol. % ethanol) (-Δ-), chestnut astringent skin extract (75 vol. % aqueous ethanol) (-▪-), chestnut astringent skin extract (50 vol. % aqueous ethanol) (-□-), and chestnut astringent skin extract (25 vol. % aqueous ethanol) (-●-) inhibited sucrase activity concentration-dependently, demonstrating that the extracts have α-glucosidase (sucrase) inhibitory activity.

(3-3) Table 3 shows the 50% inhibitory concentrations (mg/ml) of the chestnut astringent skin extracts (Preparation Examples 1 to 5) and hot water extract of guava leaves (Comparative Preparation Example) against sucrase activity. The hot extract of guava leaves is known to have α-glucosidase (sucrase) inhibitory activity (“Food Science & Business”, offprint from Nikkei Biotechnology & Business, pp. 108-111, 2003).

TABLE 3
Fifty percentage inhibitory concentrations of the extracts
for sucrase.

| Prep. | | 50% Inhibitory | |
|---|---|---|---|
| Ex. | Chestnut astringent skin extract | concentration | |
| 1 | 100 vol. % ethanol | 0.69 | mg/ml |
| 2 | 75 vol. % aqueous ethanol | 0.65 | mg/ml |
| 3 | 50 vol. % aqueous ethanol | 0.83 | mg/ml |
| 4 | 25 vol. % aqueous ethanol | 0.86 | mg/ml |
| 5 | water | 1 | mg/ml [symbol] |
| Comp. | Guava leaf extract | 0.43 | mg/ml |

Table 3 reveals that, like the chestnut astringent skin extracts exhibited α-glucosidase (sucrase) inhibitory activity equivalent to or lower than that of the hot water extract of guava leaves.

### Experiment 3

### Carbohydrate Tolerance Test on Normal Rats

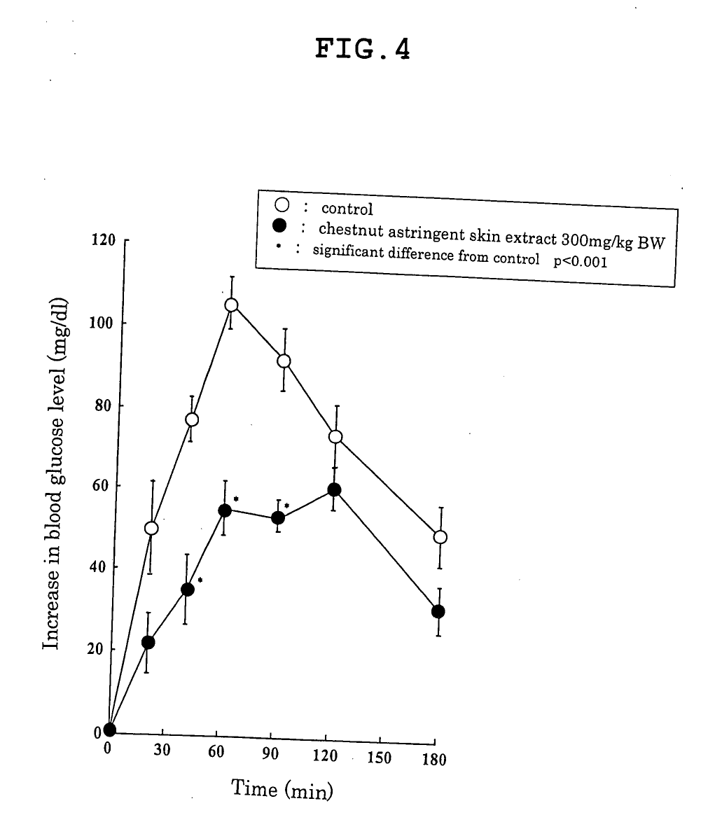

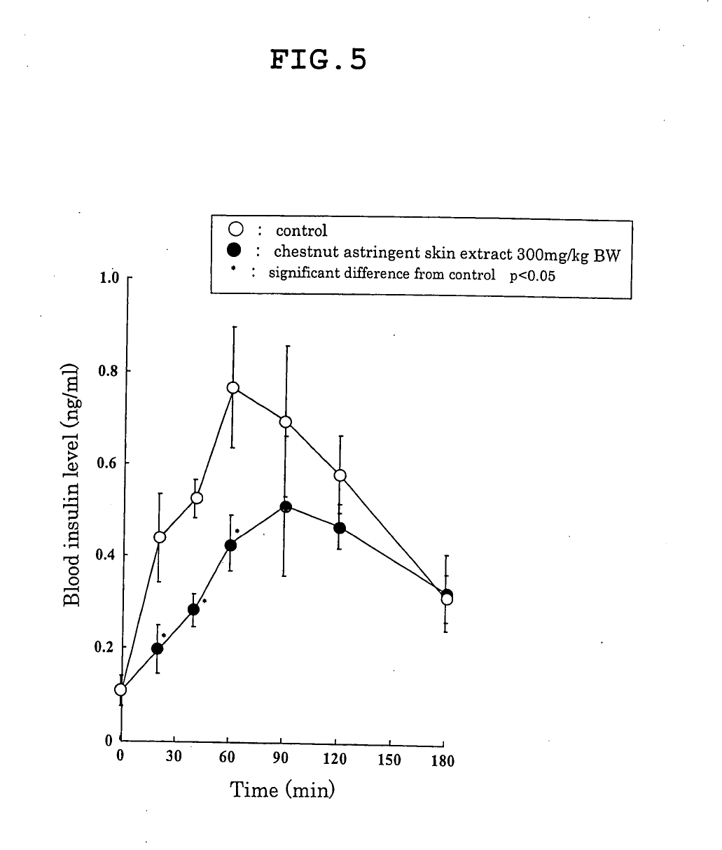

Male Wister rats weighing 150 g (Japan Clea Inc.) were preliminarily fed for 1 week, and those then weighing 180 g to 230 g were subjected to the following carbohydrate tolerance test (n=8 per group). Specifically, the rats were fasted for 12 hours, and the chestnut astringent skin extract (75 vol. % aqueous ethanol) (300 mg/kg body weight -●-) prepared in Preparation Example 2 and starch (2 g/kg body weight) were administered at the same time to the rats through a gastric tube. Blood samples were collected from the caudal vessel 0, 20, 40, 60, 90, 120 and 180 minutes after the administration, and the blood glucose levels (mg/dl) of the samples were measured to examine the changes in blood glucose levels, using a Glucocard (DIAmeter-α, ARKRAY,INC). The blood insulin levels (ng/ml) of the samples also were measured to examine the changes in blood insulin levels, using a Morinaga Insulin Kit (Morinaga Co. Ltd.). As a control test, rats were fasted for 12 hours and given only starch (2 g/kg body weight), without the chestnut astringent skin extract, through a gastric tube, and the blood glucose levels (mg/dl) and the bood insulin levels were measured at the same time points and under the same conditions as above (-◯-). FIGS. 4 and 5 show these results. The ordinate of FIG. 4 represents the increase in blood glucose levels (mg/dl) over the blood glucose levels before the administration of the test substances. The ordinate of FIG. 5 represents the increase in blood insulin levels (ng/ml) over the blood insulin levels before the administration of the test substances.

In the control rats, the blood glucose level and the blood insulin level rapidly increased until 60 minutes after the administration, whereas in the rats given the chestnut astringent skin extract, the increase in blood glucose level and the blood insulin level were remarkably suppressed depending on the concentration of the chestnut astringent skin extract administered.

This is presumably because the chestnut astringent skin extract inhibits the activities of α-amylase and α-glucosidase in the body, thereby slowing down glycolysis, suppressing carbohydrate absorption and reducing postprandial increments of blood glucose. As consequence of this attenuation and flattening of postprandial glycemia, glucose-stimulated insulin secretion is diminished.

### Example 1

### Noodles

Noodles were prepared from 500 g of medium-strength flour, 30 g of salt, 500 mg of the aqueous ethanol extract of chestnut astringent skin obtained in Preparation Example 2, 3 or 4, and 200 g of water.

### Example 2

### Hamburger Patty

A hamburger patty was prepared from 22.5 g of minced beef, 20.0 g of minced pork, 20.0 g of onion, 7.5 g of bread crumbs, 23 g of water, 2 g of salt, 1 g of sugar, 1 g of spice, 2 g of purified rapeseed oil, and 1 g of the aqueous ethanol extract of chestnut astringent skin extract obtained in Preparation Example 2, 3 or 4.

### Example 3

### Soft Drink

Hot water (1000 ml) was added to 10 g of black tea leaves to obtain an extract. One hundred grams of honey, 50g of lemon juice, and 1 g of the aqueous ethanol extract of chestnut astringent skin obtained in Preparation Example 2, 3 or 4 were added to the extract, to thereby obtain a soft drink.

### INDUSTRIAL APPLICABILITY

A substance having the carbohydrase inhibitory activity of the present invention (carbohydrase inhibitor) has an excellent inhibitory activity against α-amylase or α-glucosidase. Among such substances, it is clear that a carbohydrase inhibitor derived from an astringent skin of chestnut is safe for living bodies based on years of dietary experience.

Therefore, the carbohydrase inhibitor of the present invention is effective for reducing or preventing obesity by suppressing digestion and absorption of saccharides from alimentary canal. Furthermore, the carbohydrase inhibitor of the present invention can delay saccharide digestion and absorption and suppress a rise in postprandial blood glucose levels, and therefore it can be effectively used for ameliorating diabetic hyperglycemic conditions and preventing development in disorders of a diabetic patient caused by hyperglycemia.

Furthermore, a food composition comprising the carbohydrase inhibitor of the present invention is expected to prevent the development of diseases attributable to obesity caused by hyperphagia by the ability to inhibit digestion of starches and sugars contained in foods and preventing them being converted to energy. Furthermore, since the food composition of the present invention can suppress a rise in postprandial blood glucose levels by delaying digestion and absorption of saccharides, the food composition of the present invention is expected to have an effect in preventing or ameliorating diabetes. For example, by mixing the carbohydrase inhibitor of the present invention with foods containing a lot of starch, it is possible to provide foods for those who have high blood glucose levels or those who would like to reduce their obesity.

In recent years, production of peeled chestnuts has been increasing, and a large volume of chestnut testa is disposed as industrial waste. According to the present invention, such chestnut testae (astringent skin) can be effectively utilized. In particular, chestnut astringent skins contained in roast chesnuts and marron glace are edible, and therefore there is no reason to doubt its safety in living bodies.

1. A carbohydrase inhibitor comprising chestnut astringent skin extract, which is obtained by subjecting astringent skins of a chestnut to extraction using ethanol or aqueous ethanol solution.

2. The carbohydrase inhibitor according to claim 1, wherein the aqueous ethanol solution contains 5-95 vol. % of ethanol.

3. The carbohydrase inhibitor according to claim 1, wherein the carbohydrase to be inhibited is α-amylase, α-glucosidase or both of them.

4. A food composition comprising a carbohydrase inhibitor of claim 1.

5. The food composition according to claim 4, which is a beverage or a food high in carbohydrates.

6. The food composition according to claim 4, wherein the carbohydrase inhibitor is obtained by subjecting the astringent skin of chestnut to extraction using ethanol or aqueous ethanol solution.

7. The food composition according to claim 4, which comprises an effective amount of a carbohydrase inhibitor of claim 1 as an active ingredient for delaying saccharide digestion and absorption.

8. The food composition according to claim 7, which has a property of delaying saccharide digestion and absorption, and whose package has a note stating that the food composition is suitable for delaying saccharide digestion and absorption.

9. The food composition according to claim 4, which comprises an effective amount of a carbohydrase inhibitor of claim 1 as an active ingredient for reducing a postprandial rise in blood glucose or blood insulin.

10. The food composition according to claim 9, which has a property of suppressing postprandial rise in blood glucose levels or for ameliorating hyperglycemia, and whose package has a note stating that the food composition is suitable for suppressing a postprandial rise in blood glucose levels or for ameliorating hyperglycemia.

11. The food composition according to claim 4, which comprises a carbohydrase inhibitor of claim 1 as an active ingredient in an amount effective for preventing obesity.

12. The food composition according to claim 11, which has an anti-obesity effect, and whose package has a note stating that the food composition is suitable for preventing obesity.

13. A medical composition comprising a carbohydrase inhibitor of claim 1 as an active ingredient, together with a pharmaceutically acceptable carriers and/or additives.

14. The medical composition according to claim 13, which is a medicine for preventing or treating diabetes.

15. The medical composition according to claim 13, which is an anti-obesity medicine.

16. A method for suppressing a postprandial rise in blood glucose levels or ameliorating hyperglycemia of a subject, comprising injecting or otherwise administering a carbohydrase inhibitor of claim 1 to the subject.

17. A method for preventing or reducing obesity comprising injecting or otherwise administering a carbohydrase inhibitor of claim 1 to the subject.

18. Use of a carbohydrase inhibitor according to claim 1, for producing a composition for delaying saccharide digestion and absorption, a composition for suppressing a postprandial rise in blood glucose levels, a composition for ameliorating hyperglycemia, or an anti-obesity composition.
